(19) United States
(12) Patent Application Publication
(10) Pub. No.: US 2024/0065766 A1
(43) Pub. Date: Feb. 29, 2024

(54) COMPUTER-ASSISTED SURGICAL PLANNING

(71) Applicant: Howmedica Osteonics Corp., Mahwah, NJ (US)

(72) Inventors: Jean Chaoui, Lyon (FR); Manuel Jean-Marie Urvoy, Brest (FR); Emile Ducrocq, Brest (FR)

(21) Appl. No.: 18/275,381

(22) PCT Filed: Jan. 28, 2022
(86) PCT No.: PCT/US2022/014192

§ 371 Date: Aug. 1, 2023

Related U.S. Application Data
(60) Provisional application No. 63/146,278, filed on Feb. 5, 2021.

(51) Int. Cl. A61B 34/10; A61B 34/00; A61F 2/40; G16H 50/50

(52) U.S. Cl. CPC A61B 34/10; A61B 34/25; A61F 2/4081; G16H 50/50; A61B 2034/102; A61B 2034/105; A61B 2034/107; A61B 2034/108; A61B 2034/258

(57) ABSTRACT

A method comprises obtaining, by a computing system, one or more surgeon preference parameters that specify values of one or more surgical parameters, wherein the surgical parameters include one or more positioning parameters for a glenoid implant to be attached to a glenoid fossa of a patient during a surgery; determining, by the computing system, based on one or more anatomic parameters of the patient and the surgeon preference parameters, one or more suggested surgical options, each of the surgical options corresponding to a. different combination of the positioning parameters for the glenoid implant and types of glenoid implant; and outputting, by the computing system, for display, the one or more suggested surgical options.

This application claims priority to U.S. Provisional Patent Application 63/146,278, filed Feb. 5, 2021, the entire content of which is incorporated by reference.

### BACKGROUND

Shoulder replacement surgery is a complicated type of orthopedic surgery. However, shoulder replacement surgery is becoming increasingly common because of its ability to alleviate pain and restore range of motion in many patients. The complexity of shoulder replacement surgery has prevented many surgeons from performing shoulder replacement surgeries, especially those surgeons who do not frequently perform shoulder replacement surgeries. Accordingly, computerized surgical planning systems have been developed to help surgeons plan complicated surgeries, such as shoulder replacement surgeries.

### SUMMARY

This disclosure describes a variety of techniques for improving computerized surgical planning systems. One challenge associated with implementing computerized surgical planning systems is how to ensure that a computerized surgical planning system generates surgical plans for shoulder replacement surgeries that are consistent with the preferences of individual surgeons. For example, a computerized surgical planning system may use a machine learning (ML) model to generate a plurality of predictions regarding various surgical options for a shoulder replacement surgery. In general, a large number of training datasets (e.g., data regarding individual surgeries) may need to be used to train the ML model. At least because of the number of cases required to train ML models, it may be impractical to train separate ML models for each individual surgeon to generate predictions based on the preferences of the individual surgeons. This may especially be the case with respect to surgeons who do not frequently perform shoulder replacement surgeries because such surgeons simply have not performed enough shoulder replacement surgeries to sufficiently train ML models. This disclosure describes techniques that may address this problem and allow a surgical planning system to generate surgical suggestions that accommodate preferences of individual surgeons, with the associated benefit of reduced storage requirements and reduced utilization of computing resources.

In one example, this disclosure describes a method comprising: obtaining, by a computing system, one or more surgeon preference parameters that specify values of one or more surgical parameters, wherein the surgical parameters include one or more positioning parameters for a glenoid implant to be attached to a glenoid fossa of a patient during a surgery; determining, by the computing system, based on one or more anatomic parameters of the patient and the surgeon preference parameters, one or more suggested surgical options, each of the surgical options corresponding to a different combination of the positioning parameters for the glenoid implant and types of the glenoid implant; and outputting, by the computing system, the one or more suggested surgical options.

In another example, this disclosure describes a computing system comprising: a memory configured to store one or more surgeon preference parameters that specify values of one or more surgical parameters, wherein the surgical parameters include one or more positioning parameters for a glenoid implant to be attached to a glenoid fossa of a patient during a surgery; one or more processors implemented in circuitry, the one or more processors configured to: determine, based on one or more anatomic parameters of the patient and the surgeon preference parameters, one or more suggested surgical options, each of the surgical options corresponding to a different combination of the positioning parameters for the glenoid implant and types of glenoid implant; and output, for display, the one or more suggested surgical options.

In other examples, this disclosure describes a computing system comprising means for performing the methods of this disclosure and a computer-readable storage medium having instructions stored thereon that when executed cause one or more processors of a computing system to perform the methods of this disclosure.

The details of various examples of the disclosure are set forth in the accompanying drawings and the description below. Various features, objects, and advantages will be apparent from the description, drawings, and claims.

### BRIEF DESCRIPTION OF THE DRAWINGS

FIG. 1 is a block diagram of a surgical assistance system in accordance with one or more techniques of this disclosure.

FIG. 2 is a block diagram illustrating example details of a surgical planning system, in accordance with one or more techniques of this disclosure.

FIG. 3 is a conceptual diagram illustrating an example surgical planning user interface, in accordance with one or more techniques of this disclosure.

FIG. 4A is a conceptual diagram illustrating an example surgical planning user interface for selecting surgeon preference parameters for an anatomic shoulder replacement surgery, in accordance with one or more techniques of this disclosure.

FIG. 4B is a conceptual diagram illustrating an example surgical planning user interface for selecting surgeon preference parameters for a reverse shoulder replacement surgery, in accordance with one or more techniques of this disclosure.

FIG. 5 is a conceptual diagram illustrating an example surgical planning user interface showing surgical suggestions for an anatomic shoulder replacement surgery, in accordance with one or more techniques of this disclosure.

FIG. 6 is a conceptual diagram illustrating an example surgical planning user interface showing surgical suggestions for a reverse shoulder replacement surgery, in accordance with one or more techniques of this disclosure.

FIG. 7 is a flowchart illustrating an example operation of the surgical planning system, in accordance with one or more techniques of this disclosure.

FIG. 8 is a flowchart illustrating an example operation of a parameter prediction unit to determine one or more suggested surgical options for a glenoid implant, in accordance with one or more techniques of this disclosure.

### DETAILED DESCRIPTION

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein.

The drawings show and describe various examples of this disclosure. In the following description, numerous details are set forth. However, it will be understood by those skilled in the art that the present invention may be practiced without these details and that numerous variations or modifications from the described examples may be possible.

This disclosure describes systems and methods associated with planning a surgery. In other words, this disclosure describes techniques for automated planning of surgeries. A surgical plan, e.g., a surgical plan generated by the BLUEPRINT T, system produced by Wright Medical Group NV or another surgical planning platform, may include a variety of information regarding a surgery. For example, a surgical plan may include information regarding steps to be performed on a patient by a user, such as a surgeon. Example steps may include, for example, bone or tissue preparation steps and/or steps for selection, modification and/or placement of implant components, such as prosthetics, and associated hardware or media. Furthermore, information in a surgical plan may include, in various examples, dimensions, shapes, angles, surface contours, and/or orientations of implant components to be selected or modified by users, dimensions, shapes, angles, surface contours and/or orientations to be defined in bone or tissue by the user in bone or tissue preparation steps, and/or positions, axes, planes, angle and/or entry points defining placement of implant components by the user relative to patient bone or tissue. Information such as dimensions, shapes, angles, surface contours, and/or orientations of anatomical features of the patient may be derived from analysis of imaging (e.g., x-ray. CT, MRI, ultrasound or other images), direct observation, or other techniques.

As described herein, a computing system may obtain one or more surgeon preference parameters that specify values of one or more surgical parameters. The surgical parameters may include one or more positioning parameters for a glenoid implant to be attached to a glenoid fossa of a patient during a surgery. Additionally, the computing system may determine, based on one or more anatomic parameters of the patient and the surgeon preference parameters, one or more suggested surgical options for attaching a glenoid implant to the glenoid fossa of the patient during the surgery. Each of the surgical options corresponds to a different combination of values of the surgical parameters. The computing system may output, for display, the one or more suggested surgical options.

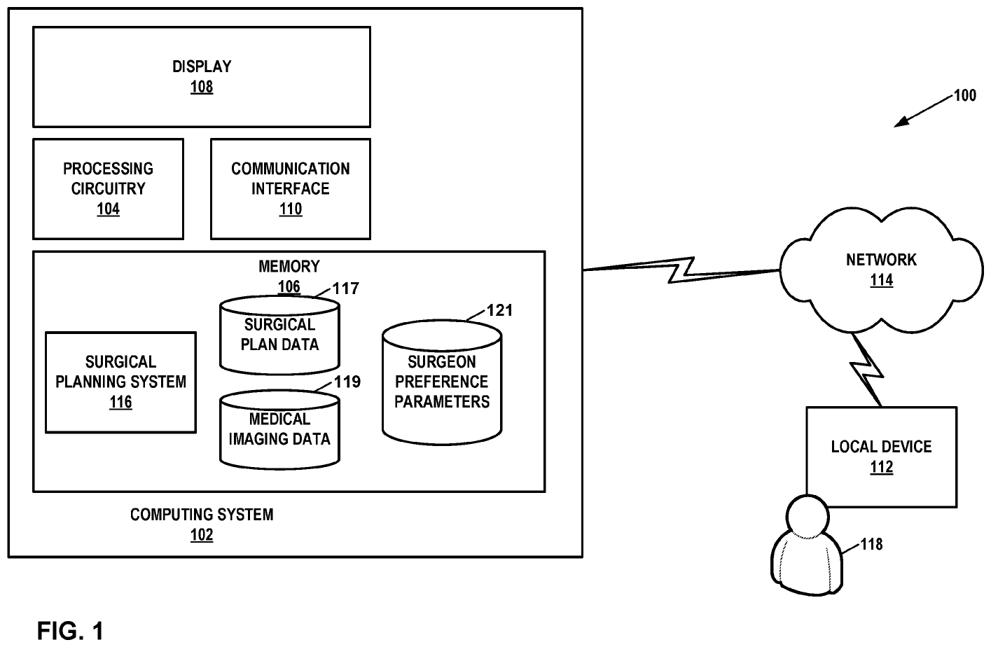

FIG. 1 is a block diagram illustrating an example surgical assistance system 100 in accordance with one or more techniques of this disclosure. In the example of FIG. 1, surgical assistance system 100 includes a computing system 102, which is an example of a computing system configured to perform one or more example techniques described in this disclosure. Computing system 102 may include various types of computing devices, such as server computers, personal computers, smartphones, tablet computers, laptop computers, and other types of computing devices. Computing system 102 includes processing circuitry 104, memory 106, a display 108, and a communication interface 110. Display 108 may be optional, such as in examples where computing system 102 comprises a server computer. Additionally, in the example of FIG. 1, surgical assistance system 100 includes a local device 112 and a communication network 114.

Examples of processing circuitry 104 include one or more microprocessors, digital signal processors (DSPs), application specific integrated circuits (ASICs), field programmable gate arrays (FPGAs), hardware, or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more units may be distinct circuit blocks (fixed-function or programmable), and in some examples, the one or more units may be integrated circuits.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, memory 106 may store the object code of the software that processing circuitry 104 receives and executes, or another memory within processing circuitry 104 (not shown) may store such instructions. Examples of the software include software designed for surgical planning. Processing circuitry 104 may perform the actions ascribed in this disclosure to computing system 102.

Memory 106 may store various types of data used by processing circuitry 104. For example, memory 106 may store data regarding one or more surgical plans. Memory 106 may be formed by any of a variety of memory devices, such as dynamic random access memory (DRAM), including synchronous DRAM (SDRAM), magnetoresistive RAM (MRAM), resistive RAM (RRAM), hard disk drives, optical discs, or other types of non-transitory computer-readable media. Examples of display 108 may include a liquid crystal display (LCD), a plasma display, an organic light emitting diode (OLED) display, or another type of display device.

Furthermore, in the example of FIG. 1, memory 106 may include computer-readable instructions that, when executed by processing circuitry 104, cause computing system 102 to provide a surgical planning system 116. In some examples, some or all of the instructions of surgical planning system 116 are stored on local device 112 and/or executed by processing circuitry of local device 112. In other examples, some or all of the instructions of surgical planning system 116 are stored on computing system 102 and/or executed by processing circuitry of computing system 102. In some examples, local device 112 may be or may include a mixed reality (MR) visualization device. For ease of explanation, this disclosure may simply describe actions performed by computing system 102 and/or local device 112 when processing circuitry 104 and/or processing circuitry of local device 112 executes instructions of surgical planning system 116 as being performed by surgical planning system 116, with the understanding that processing operations may be performed by processing circuitry of computing system 102, local device 112, or a combination both, or by or in combination with other processing circuitry, including processing circuitry associated with one or more cloud servers and/or one or more other remote computing devices. In the example of FIG. 1, memory 106 may also include surgical plan data 117, medical imaging data 119, and surgeon preference parameters 121.

Communication interface 110 allows computing system 102 to output data and instructions to and receive data and instructions from local device 112 and/or other devices via a network 114. Communication interface 110 may comprise hardware circuitry that enables computing system 102 to communicate (e.g., wirelessly or using wires) to other computing systems and devices, such as MR visualization device 112. Network 114 may include various types of communication networks including one or more wide-area networks, such as the Internet, local area networks, and so on. In some examples, network 114 may include wired and/or wireless communication links.

Local device 112 may be a computing device used by a user 118. In other examples, user 118 may directly use a computing device of computing system 102. In such examples, user 118 may view content displayed on display 108. In some examples, local device 112 is a personal computer, smartphone, tablet computer, laptop computer, or another type of computing device. In some examples, local device 112 is a mixed reality (MR) visualization device. An MR visualization device may use various visualization techniques to display image content to user 118, who may be a surgeon. For instance, an MR visualization device may include a holographic projector or other type of device for presenting MR scenes. In some examples where local device 112 is an MR visualization device, local device 112 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting user 118 to view actual objects in a real-world scene, i.e., in a real-world environment, through the holographic lenses.

As mentioned above, memory 106 may include computer-readable instructions that, when executed by processing circuitry 104, cause computing system 102 to provide a surgical planning system 116. Surgical planning system 116 is configured to help a surgeon plan a surgery, such as an anatomic shoulder replacement surgery or a reverse shoulder replacement surgery. In an anatomic shoulder replacement surgery, a surgeon implants a cup-shaped glenoid implant on a glenoid fossa of a patient's scapula and a ball-shaped humeral implant on the proximal end of the patient's humerus. In a reverse shoulder replacement surgery, a surgeon implants a ball-shaped glenoid implant on the glenoid fossa of the patient's scapula and a cup-shaped humeral implant on the proximal end of the patient's humerus.

In either an anatomic shoulder replacement surgery or a reverse shoulder replacement surgery, the surgeon can select from among various different types of glenoid implants and humeral implants. For example, the surgeon can select among glenoid implants with keels, glenoid implants with pegs, or other types of glenoid implants. Moreover, the surgeon can select among various sizes within each type of glenoid implant. Furthermore, the surgeon can select among stemmed humeral implants, stemless humeral implants, or other types of humeral implants. Similarly, the surgeon can select among various sizes within each type of glenoid implant. For any one glenoid or humeral implant, the surgeon can select among various placement parameters for the glenoid or humeral implants. For example, the surgeon can select among various angles at which to seat the glenoid or humeral implants. In some examples, the surgeon can select among various bone preparation angles, depths, and positions.

Given that there are numerous types of implants and various available placement parameters, it may be difficult for surgeons to choose which types of implants and which placement parameters to use for a specific patient. Accordingly, surgical planning system 116 may generate suggested surgical options regarding types of implants and placement parameters to use for specific patients when the surgeon is planning shoulder replacement surgeries for the patients. When planning a shoulder replacement surgery, the surgeon may select from among the suggested surgical options generated by surgical planning system 116 or select other types of implants and/or surgical parameters. In other words, the surgeon is not limited to the suggested surgical options generated by surgical planning system 116.

Individual surgeons may have specific preferences with respect to types of implants and placement parameters. For example, a surgeon may prefer to always use pegged glenoid implants instead of keeled glenoid implants because they feel that patients have lower revision rates with pegged glenoid implants than keeled glenoid implants. In another example, a surgeon may prefer to have implant retroversion angles for glenoid implants be no greater than 10°.

Ignoring the preferences of surgeons may result in surgical planning system 116 generating suggested surgical options that surgeons will not use. This presents a significant limit on the utility of surgical planning system 116 generating suggestions at all. One approach to addressing this problem is to train machine learning (ML) models based on surgeries performed according to the preferences of individual surgeons. However, the surgeon may not have completed enough surgeries for there to be sufficient training data to train an ML model for the surgeon. Without sufficient training data, the ML model for a surgeon may generate poor suggestions. Moreover, implementing different ML models for different surgeons may consume considerable processing power and storage space.

The techniques of this disclosure may address this issue. As described herein, surgical planning system 116 may obtain one or more surgeon preference parameters 121 that specify ranges of surgical parameters, such as glenoid implant types and positioning parameters for a glenoid implant to be attached to a glenoid of a patient during a surgery. Furthermore, surgical planning system 116 may determine, based on one or more anatomic parameters of the patient and the surgeon preference parameters, one or more suggested surgical options. The suggested surgical options may correspond to different combinations of the positioning parameters for the implant and types of glenoid implant.

Examples of how surgical planning system 116 may perform automated planning to determine the suggested surgical options based on the one or more anatomic parameters and the surgeon preference parameters are described in greater detail below. Surgical planning system 116 may output the one or more suggested surgical options for display. In some examples, surgical planning system 116 may receive an indication of user input from the surgeon to select one of the suggestions or select alternative implant types or placement parameters. Surgical planning system 116 may store the selected implant types and/or placement parameters in surgical plan data 117.

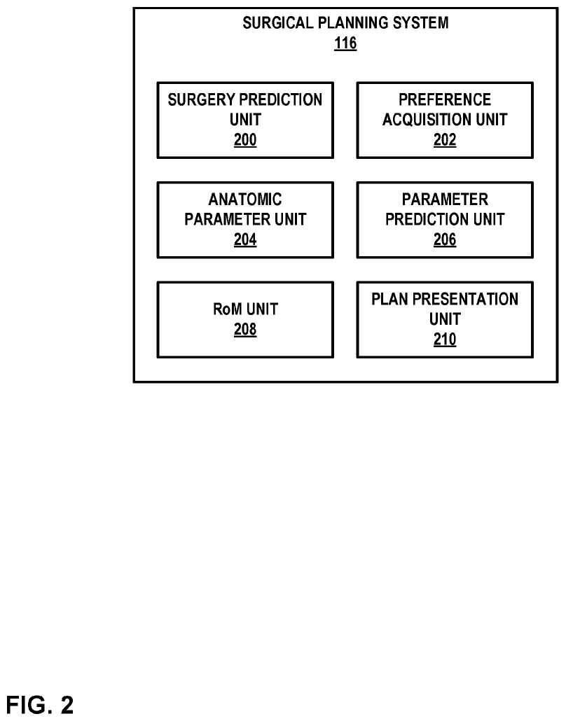

FIG. 2 is a block diagram illustrating example details of surgical planning system 116, in accordance with one or more techniques of this disclosure. In the example of FIG. 2, surgical planning system 116 includes a surgery prediction unit 200, a preference acquisition unit 202, an anatomic parameter unit 204, a parameter prediction unit 206, a range of motion (RoM) unit 208, and a plan presentation unit 210. In other examples, surgical planning system 116 may include more, fewer, or different units. Surgery prediction unit 200, preference acquisition unit 202, anatomic parameter unit 204, parameter prediction unit 206, RoM unit 208, and plan presentation unit 210 may be implemented in software executed by programmable processing circuitry. In some examples, one or more of surgery prediction unit 200, preference acquisition unit 202, anatomic parameter unit 204, parameter prediction unit 206, RoM unit 208, and plan presentation unit 210 may be at least partially implemented using special-purpose hardware. Surgery prediction unit 200, preference acquisition unit 202, anatomic parameter unit 204, parameter prediction unit 206, RoM unit 208 may work together to generate computer-assisted predictions.

Surgery prediction unit 200 may generate a prediction regarding whether to perform an anatomic shoulder replacement surgery or a reverse shoulder replacement surgery. For example, surgery prediction unit 200 may generate a first confidence value indicating a level of confidence (e.g., an estimated probability) that a set of reference surgeons would select an anatomic shoulder replacement surgery for a patient and a second confidence value indicating a level of confidence that the set of reference surgeons would select a reverse shoulder replacement surgery for the patient. In this example, surgery prediction unit 200 may output an indication of whichever of the anatomic shoulder replacement surgery and the reverse shoulder replacement surgery has a greater confidence score.

Surgery prediction unit 200 may be implemented in one of a variety of ways. For example, surgery prediction unit 200 may be implemented using one or more artificial intelligence systems, such as a combination of one or more artificial neural networks, support vector machines (SVMs), decision tree networks, random forests, naïve Bayesian networks, and so on. Surgery prediction unit 200 may generate the prediction regarding whether to perform an anatomic shoulder replacement surgery or a reverse shoulder replacement surgery based on a set of input data. Example types of input data for surgery prediction unit 200 may include patient data such as the age of the patient, a diagnosis of a condition of the patient (e.g., massive rotator cuff tear, osteoarthritis, etc.), a gender of the patient, a glenoid orientation of the patient, a glenoid sphere radius of the patient, a glenoid version of the patient, a glenoid inclination of the patient, a humerus subluxation of the patient, a glenoid direction of the patient, a glenoid area of the patient, and/or other types of data regarding the patient.

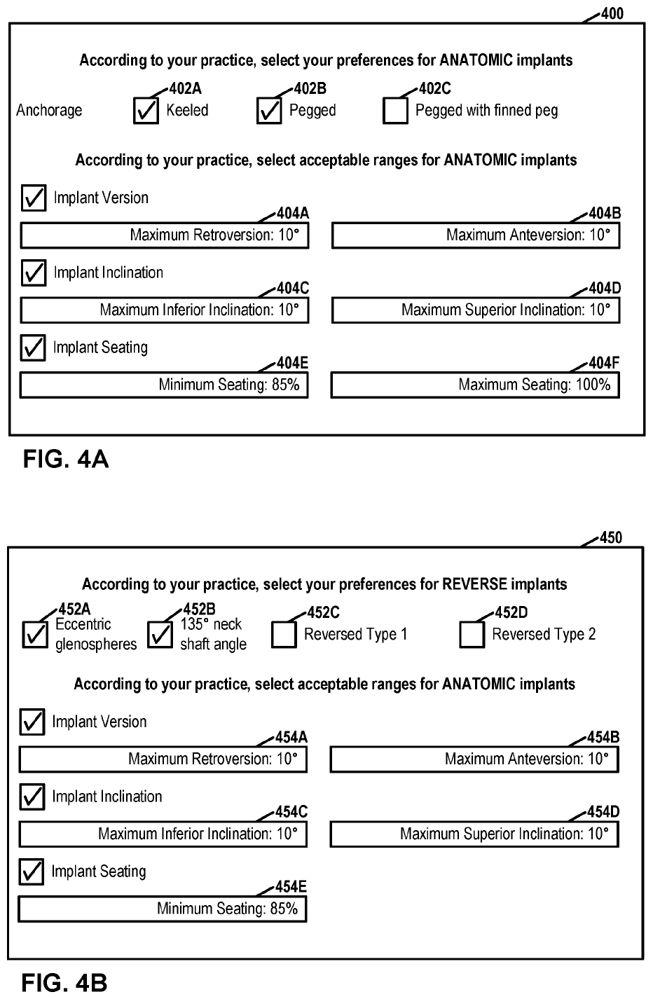

Preference acquisition unit 202 is configured to acquire surgeon preference parameters for a surgeon. Preference acquisition unit 202 may store acquired surgeon preference parameters as surgeon preference parameters 121. In some examples, the surgeon preference parameters may be specific to a surgery for a specific patient. In some examples, the surgeon preference parameters may be common across all patients treated by the surgeon. Preference acquisition unit 202 may output a user interface for selecting surgeon preference parameters. FIG. 4A, which is described in greater detail below, illustrates an example user interface for selecting surgeon preference parameters for an anatomic shoulder replacement surgery. FIG. 4B, which is described in greater detail below, illustrates an example user interface for selecting surgeon preference parameters for a reverse shoulder replacement surgery.

With respect to the example of FIG. 2, anatomic parameter unit 204 is configured to determine anatomic parameters of the patient. In some examples, anatomic parameter unit 204 is configured to determine one or more anatomic parameters of the patient based on indications of input of the anatomic parameters from user 118. In some examples, anatomic parameter unit 204 is configured to determine one or more anatomic parameters of the patient based on medical imaging data 119. Medical imaging data 119 may include x-ray images, computed tomography (CT) images, magnetic resonance imaging (MRI) images, and so on. Example types of anatomic parameters of the patient may include a glenoid sphere radius, a glenoid orientation, a reverse shoulder angle, a critical shoulder angle, a glenoid rotation angle, a coracoid process angle, an infra glenoid tubercle angle, an acromion index, an acromio-humeral space, a humeral subluxation percentage, a humeral head radius, a humeral direction, a humeral orientation, a humeral head center-to-glenoid center distance, a Giannotti cortical index for the humerus, a Tingart cortical thickness of the humerus, a proximal diaphysis bone density value for the humerus, a metaphysis spongious bone density value for the humerus, a metaphysis cortical bone density value for the humerus, bone density values for the scapula, glenoid version, glenoid inclination, humerus subluxation, and/or other types of information regarding the anatomy of the patient.

In some examples, to determine one or more of the anatomic parameters of the patient based on medical imaging data 119, anatomic parameter unit 204 may generate 3-dimensional (3D) models of the bones (e.g., scapula, humerus, etc.) of the patient based on medical imaging data 119. Additionally, anatomic parameter unit 204 may perform processes to identify specific landmarks in the 3D models of the bones. The landmarks are positions in 3D space that are on or within the 3D models of the bones. Anatomic parameter unit 204 may then use the positions of the landmarks in 3D space to calculate one or more of the anatomic parameters. For example, to calculate the critical shoulder angle, anatomic parameter unit 204 may determine an angle between (i) a line from a most-superior point (i.e., a first landmark) on a border of a glenoid fossa of the patient to a most-inferior point (i.e., a second landmark) on the border of the glenoid fossa of the patient, and (ii) a line from the most-inferior point on the border of the glenoid fossa of the patient to a most-lateral point (i.e., a third landmark) on an acromion of the scapula of the patient. Anatomic parameter unit 204 may use one or more types of algorithms to identify the landmarks. For example, anatomic parameter unit 204 may use a hill-climbing algorithm to identify specific landmarks, such as points on the border of the glenoid fossa of the patient.

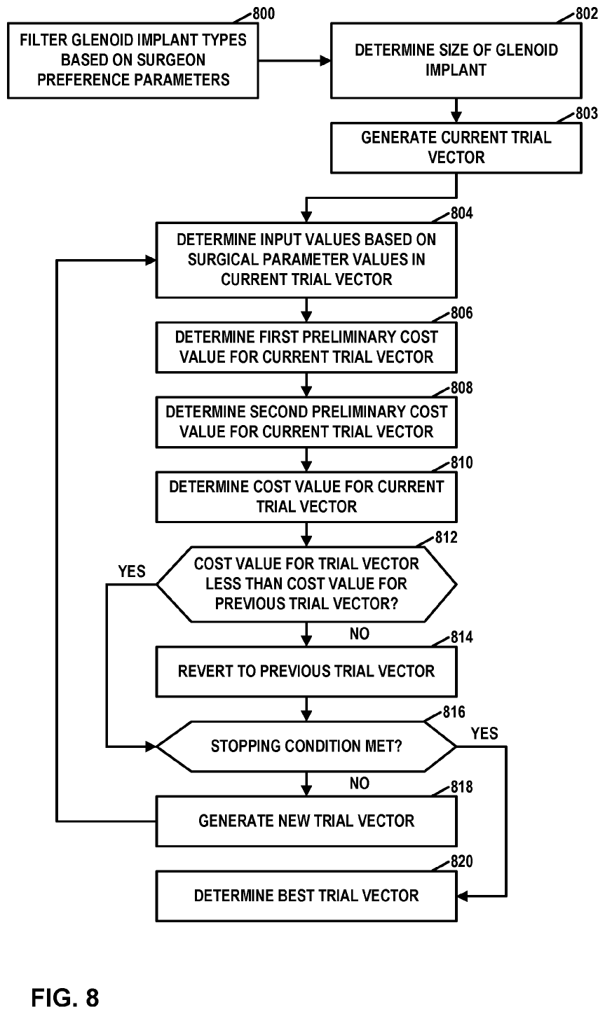

Parameter prediction unit 206 may determine one or more suggested surgical options based on the one or more anatomic parameters of the patient and the surgeon preference parameters. The suggested surgical options may correspond to different combinations of glenoid implants, positioning parameters, and/or bone preparation parameters for glenoid implants. Parameter prediction unit 206 may perform a process to determine the suggested surgical options that includes several stages. In a first stage, parameter prediction unit 206 may filter glenoid implant types based on surgeon preference parameters. In a second stage, parameter prediction unit 206 may determine a size of the glenoid implant. In a third stage, parameter prediction unit 206 may use a cost function to determine cost values for trial vectors. Each trial vector is a set of surgical parameter values, such as positioning parameters and/or glenoid implant type. FIG. 8, which is described in greater detail below, describes an example process to determine suggested surgical options for a glenoid implant.

RoM unit 208 may determine a range of motion (RoM) of the patient's shoulder for one or more of the suggested surgical options. RoM unit 208 may determine a range of motion for a suggested surgical option using a combined 3D model of the patient's scapula, humerus (with humeral implant), and the glenoid implant attached to the patient's scapula with the surgical parameters corresponding to the suggested surgical option. RoM unit 208 may then move the 3D model of the humerus relative to the 3D models of the scapula and glenoid implant along one or more axes of motion. For each axis of motion, RoM unit 208 may then detect the angles at which the model of the humerus collides with the model of the scapula. These collisions represent the outermost ends of the ranges of motion for the axes of motion.

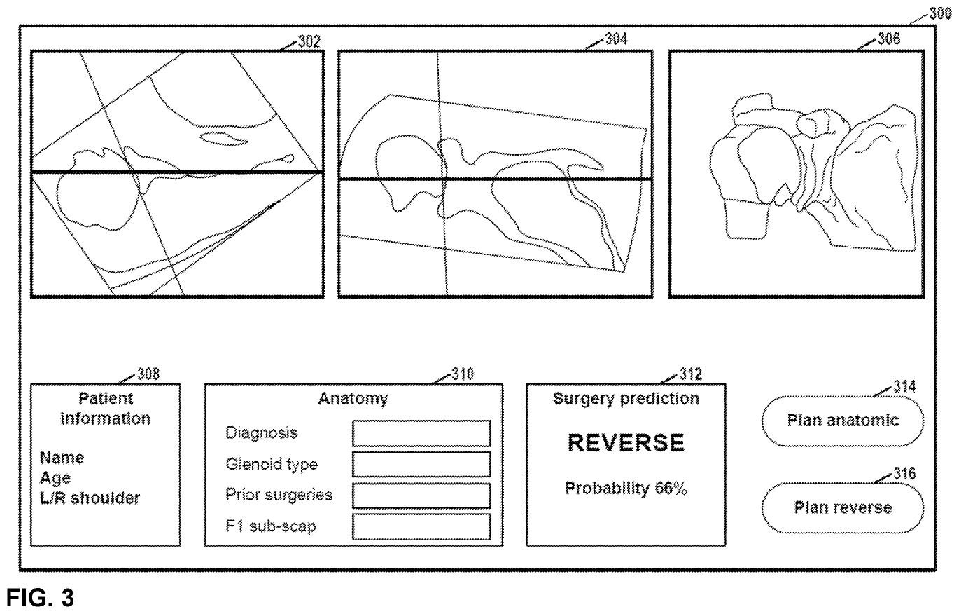

FIG. 3 is a conceptual diagram illustrating an example surgical planning user interface 300, in accordance with one or more techniques of this disclosure. Plan presentation unit 210 (FIG. 2) may generate user interface 300 for display (e.g., on display 108 or local device 112 (FIG. 1)). User 118 may use interface 300 as part of a process to plan a shoulder replacement surgery. In the example of FIG. 3, user interface 300 includes a superior view 302, a frontal view 304, and a bone model 306. In this example, superior view 302 is an x-ray image of a shoulder of a patient from a superior perspective (i.e., looking in the inferior direction from a superior position). In this example, frontal view 304 is an x-ray image of the shoulder of the patient from an anterior perspective (i.e., looking in the posterior direction from an anterior position). In this example, model 306 is a 3-dimensional model of the bone of the patient's shoulder. Superior view 302, frontal view 304, and model 306 may help user 118 visualize the patient's shoulder for purposes of planning a shoulder replacement surgery on the patient's shoulder.

Additionally, surgical planning interface 300 includes a patient information field 308, a patient anatomy field 310, a surgery prediction field 312, a “plan anatomic” button 314, and a “plan reverse” button 316. Patient information field 308 includes name information, age information, and information about whether the surgery is being planned on the patient's left or right shoulder. Patient anatomy field 310 includes information about the patient's diagnosis, glenoid type, prior surgeries, and F1 sub-scapularis footprint (F1 sub-scap). Surgery prediction field 312 may include an indication of a predicted type of shoulder replacement surgery for the patient. In the example of FIG. 3, surgery prediction field 312 indicates that the predicted type of shoulder replacement surgery for the patient is a reverse shoulder replacement surgery with a probability of 66%. In other words, given the information about the patient, a majority of the surgeons associated with the training data would perform the predicted type of shoulder replacement surgery and a confidence level in the prediction is 66%. Surgery prediction unit 200 (FIG. 2) may determine the predicted type of shoulder replacement surgery, e.g., as described elsewhere in this disclosure.

User 118 may initiate a process to plan an anatomic shoulder replacement surgery by selecting “plan anatomic” button 314. User 118 may initiate a process to plan a reverse shoulder replacement surgery by selecting “plan reverse” button 316. Note that, in some examples, user 118 does not need to, i.e., is not required to, select the type of shoulder replacement surgery indicated in surgery prediction field 312, and may instead select another type of shoulder replacement surgery that is not indicated.

FIG. 4A is a conceptual diagram illustrating an example surgical planning user interface 400 for selecting surgeon preference parameters for an anatomic shoulder replacement surgery, in accordance with one or more techniques of this disclosure. In some examples, preference acquisition unit 202 (FIG. 2) may present user interface 400 in response to receiving an indication of user input to select “plan anatomic” button 314 (FIG. 3).

In the example of FIG. 4A, user interface 400 includes anchorage checkboxes 402A-402C (collectively. “anchorage checkboxes 402”). Anchorage checkbox 402A corresponds to glenoid implants having keeled anchorages. Anchorage checkbox 402B corresponds to glenoid implants having pegged anchorages. Anchorage checkbox 402C corresponds to glenoid implants having pegged anchorages that include one or more finned pegs. In other examples, anchorage checkboxes 402 may correspond to other types of anchorage arrangements for glenoid implants. User 118 (e.g., surgeon) may use anchorage checkboxes 402 to indicate which types of anchorage arrangements for glenoid implants may be used by parameter prediction unit 206 to determine suggested surgical options.

Furthermore, in the example of FIG. 4A, user interface 400 includes range selection features 404A-404F (collectively, “range selection features 404”). Range selection feature 404A corresponds to a maximum retroversion angle of the glenoid implant. Range selection feature 404B corresponds to a maximum anteversion angle of the glenoid implant. Range selection feature 404C corresponds to a maximum inferior inclination angle of the glenoid implant. Range selection feature 404D corresponds to a maximum superior inclination angle of the glenoid implant. Range selection feature 404E corresponds to a minimum seating percentage of the glenoid implant. A seating percentage is a percentage of an area of a seating surface of the implant that is in contact with (i.e., seats on) the bone. Range selection feature 404F corresponds to a maximum seating percentage of the glenoid implant.

FIG. 4B is a conceptual diagram illustrating an example surgical planning user interface 450 for selecting surgeon preference parameters for an anatomic shoulder replacement surgery, in accordance with one or more techniques of this disclosure. In some examples, preference acquisition unit 202 (FIG. 2) may present user interface 450 in response to receiving an indication of a user input to select “plan reverse” button 316 (FIG. 3).

In the example of FIG. 4B, user interface 450 includes checkboxes 452A-452D (collectively, “checkboxes 452”). Checkboxes 452 correspond to types of implants that the surgeon is willing to use in a reverse shoulder replacement surgery. Checkbox 452A corresponds to eccentric glenospheres. Checkbox 452B corresponds to glenoid implants with 135° neck shaft angles. Checkbox 452C corresponds to a first type of glenoid implants. Checkbox 452D corresponds to a second type of glenoid implants. In other examples, checkboxes 452 may correspond to other types of implants used in reverse shoulder replacement surgeries. User 118 (e.g., surgeon) may use checkboxes 452 to indicate which types of implants may be used by parameter prediction unit 206 to determine suggested surgical options.

Furthermore, in the example of FIG. 4B, user interface 450 includes range selection features 454A-454E (collectively, “range selection features 454”). Range selection feature 454A corresponds to a maximum retroversion angle of the glenoid implant. Range selection feature 454B corresponds to a maximum anteversion angle of the glenoid implant. Range selection feature 454C corresponds to a maximum inferior inclination angle of the glenoid implant. Range selection feature 454D corresponds to a maximum superior inclination angle of the glenoid implant. Range selection feature 454E corresponds to a minimum seating percentage of the glenoid implant.

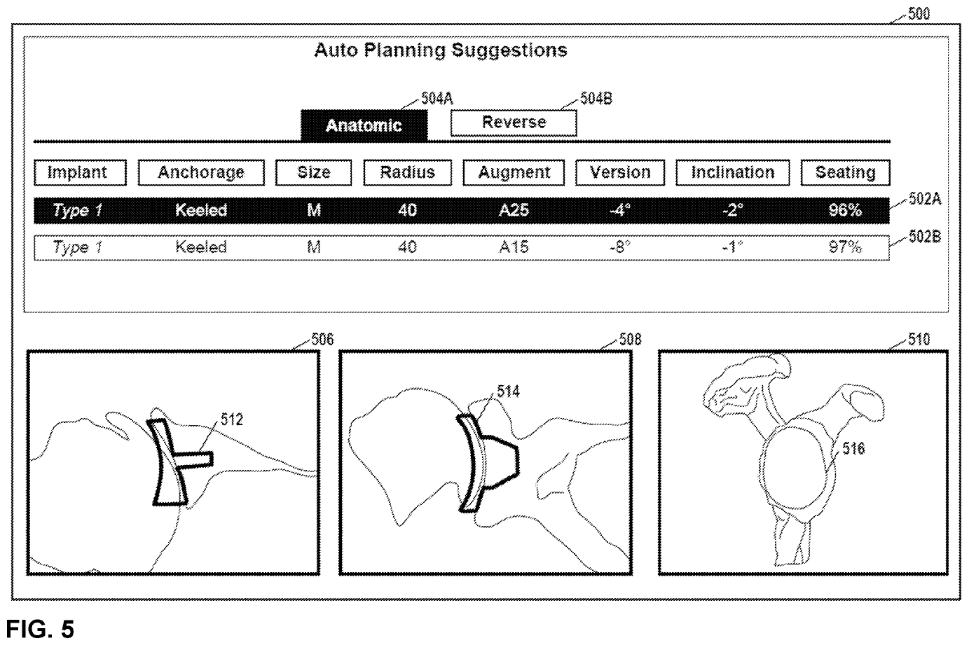

FIG. 5 is a conceptual diagram illustrating an example surgical planning user interface 500 showing surgical suggestions for an anatomic shoulder replacement surgery, in accordance with one or more techniques of this disclosure. Plan presentation unit 210 (FIG. 2) may generate user interface 500 for display (e.g., on display 108 or local device 112 (FIG. 1)). In some examples, plan presentation unit 210 may generate user interface 500 after receiving indications of user input indicating surgeon preference parameters (e.g., via user interface 300 (FIG. 3)).

In the example of FIG. 5, user interface 500 shows surgical suggestions 502A, 502B (collectively, “surgical suggestions 502”) for an anatomic shoulder replacement surgery. Each of surgical suggestions 502 indicates a type of a glenoid implant, an anchorage type of the glenoid implant, a size of the glenoid implant, a radius of a sphere of the glenoid implant, an augment of the glenoid implant, a version of the glenoid implant, an inclination of the glenoid implant, and a seating percentage of the glenoid implant. An augment of a glenoid implant is a device that compensates for high glenoid versions. In other examples, surgical suggestions 502 may indicate more, fewer, or different types of data. For instance, in some examples, surgical suggestions 502 may indicate an amount of reaming (e.g., in cubic millimeters or in millimeters). In some examples, surgical suggestions 502 may include two radii of curvature for augmented glenoid implants and a single radius of curvature for non-augmented glenoid implants. User 118 may select one of surgical suggestions 502. In the example of FIG. 5, the black background is used to indicate that surgical suggestion 502A is the selected surgical suggestion.

Furthermore, user interface 500 includes superior view 506, frontal view 508, and model 510. Superior view 506 shows an x-ray image of the shoulder of a patient from a superior perspective (i.e., looking in the inferior direction from a superior position). Superior view 506 shows an outline 512 of a glenoid implant of the type indicated by the selected surgical suggestion at the positions indicated by the selected surgical suggestion. Frontal view 508 shows an x-ray image of the shoulder of the patient from an anterior perspective (i.e., looking in the posterior direction from an anterior position). Frontal view 508 shows an outline 514 of the glenoid implant of the type indicated by the surgical suggestion at the positions indicated by the selected surgical suggestion. Model 510 shows a 3D model of the patient's scapula with the glenoid fossa 516 highlighted.

User interface 500 also includes controls 504A, 504B for switching between display of surgical suggestions for an anatomic shoulder replacement surgery and a reverse shoulder replacement surgery.

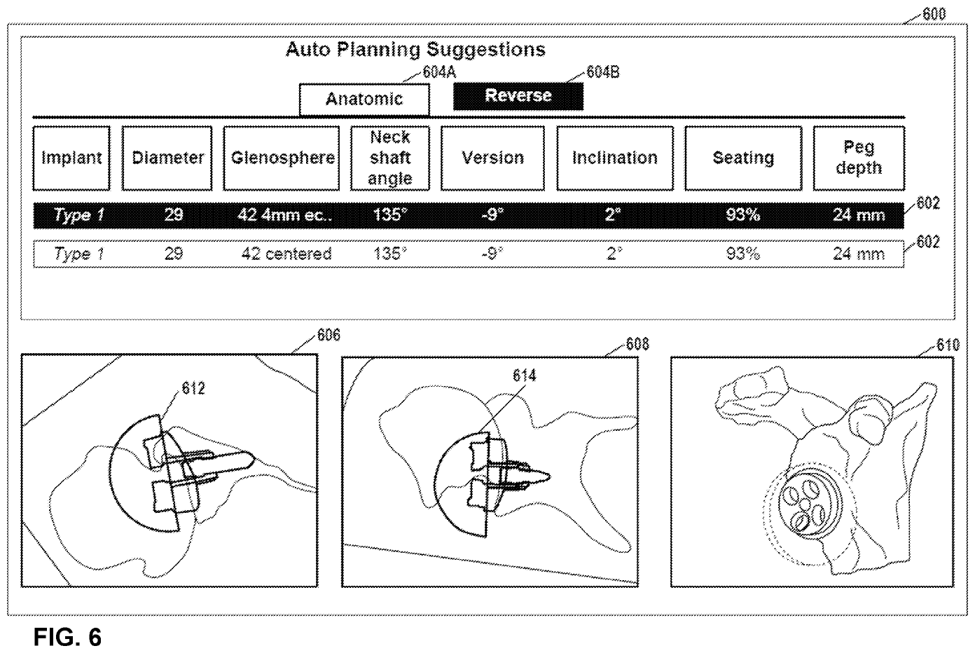

FIG. 6 is a conceptual diagram illustrating an example surgical planning user interface 600 showing surgical suggestions for a reverse shoulder replacement surgery, in accordance with one or more techniques of this disclosure. Plan presentation unit 210 (FIG. 2) may generate user interface 600 for display (e.g., on display 108 or local device 112 (FIG. 1)). In some examples, plan presentation unit 210 may generate user interface 600 after receiving indications of user input indicating surgeon preference parameters (e.g., via user interface 300 (FIG. 3)).

In the example of FIG. 6, user interface 600 shows surgical suggestions 602A, 602B (collectively, “surgical suggestions 602”) for a reverse shoulder replacement surgery. Each of surgical suggestions 602 indicates a type of a glenoid implant, a diameter of the glenoid implant, a glenosphere diameter and type (e.g., centered, eccentric, tilted, etc.) of the glenoid implant, a neck shaft angle of a corresponding humerus implant, a version of the glenoid implant, a seating percentage of the glenoid implant, and a peg depth of the glenoid implant. User 118 may select one of surgical suggestions 602. In the example of FIG. 6, the black background is used to indicate that surgical suggestion 602A is the selected surgical suggestion.

Furthermore, user interface 600 includes superior view 606, frontal view 608, and model 610. Superior view 606 shows an x-ray image of the shoulder of a patient from a superior perspective (i.e., looking in the inferior direction from a superior position). Superior view 606 shows an outline 612 of a glenoid implant of the type indicated by the selected surgical suggestion at the positions indicated by the selected surgical suggestion. Frontal view 608 shows an x-ray image of the shoulder of the patient from an anterior perspective (i.e., looking in the posterior direction from an anterior position). Frontal view 608 shows an outline 614 of the glenoid implant of the type indicated by the surgical suggestion at the positions indicated by the selected surgical suggestion. Model 610 shows a 3D model of the patient's scapula with a phantom image of a glenoid implant.

User interface 600 also includes controls 604A, 604B for switching between display of surgical suggestions for an anatomic shoulder replacement surgery and a reverse shoulder replacement surgery.

Although not shown in the example of FIG. 6, each of surgical suggestions 602 may include data indicating expected ranges of motion for surgical suggestions 602. For example, each of surgical suggestions 602 may indicate an expected angle of extension, an expected angle of flexion, an expected angle of abduction, and an expected angle of internal rotation. RoM unit 208 may determine these expected ranges of motion, e.g., in the manner described elsewhere in this disclosure.

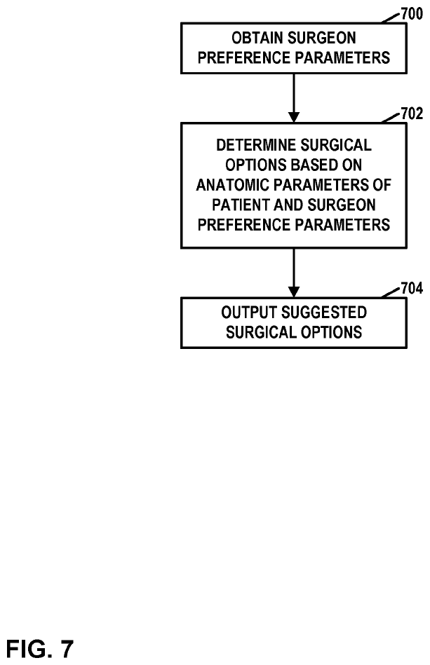

FIG. 7 is a flowchart illustrating an example operation of surgical planning system 116, in accordance with one or more techniques of this disclosure. In the example of FIG. 7, surgical planning system 116 (e.g., preference acquisition unit 202 (FIG. 2) may obtain one or more surgeon preference parameters that specify values of one or more surgical parameters (700). The surgical parameters may indicate ranges of positioning parameters for a glenoid implant to be attached to a glenoid of a patient during a surgery. For example, surgical planning system 116 may obtain the surgeon preference parameters via a user interface, such as user interface 400 (FIG. 4A) or user interface 450 (FIG. 4B).

Furthermore, in the example of FIG. 7, surgical planning system 116 (e.g., parameter prediction unit 206) may determine, based on one or more anatomic parameters of the patient and the surgeon preference parameters, one or more suggested surgical options (702). Each of the surgical options corresponds to a different combination of the positioning parameters for the glenoid implant and types of glenoid implant. FIG. 8, described in detail below, is a flowchart illustrating an example operation of a parameter prediction unit 206 to determine the one or more suggested surgical options for the glenoid implant. In some examples, as part of determining the one or more suggested surgical options, parameter prediction unit 206 may filter suggested surgical options (e.g., suggested surgical options determined using the operation of FIG. 8) to remove invalid suggested surgical options. For example, parameter prediction unit 206 may filter out suggested surgical options that include glenoid implants having anchorages that perforate a boundary of the scapula opposite the glenoid.

In some examples, surgical planning system 116 may obtain medical imaging data for the glenoid of the patient. For example, surgical planning system 116 may obtain the medical imaging data from a memory, such as memory 106 (FIG. 1), from a medical imaging machine (e.g., an x-ray machine, CT machine, etc.). The medical imaging data may include medical images and/or models of the shoulder of the patient. Surgical planning system 116 (e.g., anatomic parameter unit 204) may determine the one or more anatomic parameters of the patient based on the medical imaging data.

In the example of FIG. 7, surgical planning system 116 (e.g., plan presentation unit 210 (FIG. 2) may output the one or more suggested surgical options (704). For example, surgical planning system 116 may output the one or more suggested surgical options in a user interface, such as user interface 500 (FIG. 5) or user interface 600 (FIG. 6). In some examples, surgical planning system 116 may output the one or more suggested surgical options for display in an MR visualization. In some examples, surgical planning system 116 may output the one or more suggested surgical options for display on a conventional monitor or screen. In some examples, surgical planning system 116 may output the suggested surgical options audibly.

FIG. 8 is a flowchart illustrating an example operation of parameter prediction unit 206 to determine one or more suggested surgical options for a glenoid implant, in accordance with one or more techniques of this disclosure. In the example of FIG. 8, parameter prediction unit 206 may filter glenoid implant types based on surgeon preference parameters (800). In other words, parameter prediction unit 206 may filter out glenoid implant types based on the surgeon preference parameters to determine a set of one or more remaining glenoid implant types. When filtering glenoid implant types, parameter prediction unit 206 may start from a set of glenoid implants that includes all available glenoid implant types. For example, the glenoid implant types may include glenoid implants with keeled anchorages, glenoid implants with pegged anchorages, and glenoid implants with pegged anchorages that include one or more finned pegs. Furthermore, in this example, if the surgeon preference parameters indicate that the surgeon does not want to use glenoid implants with keeled anchorages, parameter prediction unit 206 may filter out (e.g., remove) all glenoid implants with keeled anchorages from a list of available glenoid implants.

Additionally, in the example of FIG. 8, parameter prediction unit 206 may determine a size of a glenoid implant based on the anatomical parameters of the patient (802). For instance, parameter prediction unit 206 may determine a glenoid area size (i.e., an anatomical parameter) of the glenoid fossa of the patient. In this example, the glenoid area size of the glenoid fossa is the 2-dimensional area contained within a border of the glenoid fossa. Parameter prediction unit 206 may then compare the glenoid area size of the glenoid to a set of one or more thresholds. The thresholds may correspond to sizes of glenoid implants in the list of available glenoid implants. In some examples, parameter prediction unit 206 may determine the glenoid area size, a length of a glenoid major axis, and a length of a glenoid minor axis. The glenoid major axis and the glenoid minor axis are defined by an ellipse corresponding to a boundary of the glenoid. Parameter prediction unit 206 may determine the size of the glenoid implant based on the glenoid area size, the length of the glenoid major axis, and the length of the glenoid minor axis. For instance, parameter prediction unit 206 may look up the size of the glenoid implant in a table that maps combinations of glenoid area size, length of the glenoid major axis, and the length of the glenoid minor axis to sizes of glenoid implants.

In some examples, rather than parameter prediction unit 206 automatically determining the size of the glenoid implant, parameter prediction unit 206 may receive data indicating a user-specified size of the glenoid implant. For example, parameter prediction unit 206 may receive an indication of user input of the size of the glenoid implant. In another example, parameter prediction unit 206 may receive an indication of a set of rules from user 118 that parameter prediction unit 206 may use to determine the size of the glenoid implant. In this way, user 118 may be able to choose a lower size implant in order to achieve a stronger seating for the glenoid implant. In another example, user 118 may select a particular size for the glenoid implant because user 118 may feel that anatomic parameter unit 204 has determined an incorrect glenoid area size. In another example, user 118 may select a particular size of the glenoid implant in order to avoid osteophytes.

Furthermore, parameter prediction unit 206 may generate a current trial vector (803). The trial vector is a set of surgical parameter values. The surgical parameter values are values of surgical parameters. The surgical parameters may include placement parameters and types of glenoid implants. Example placement parameters may include a version of the glenoid implant, an inclination of the glenoid implant, an anterior position of the glenoid implant, a lateral position of the glenoid implant, a superior position of the glenoid implant, and so on. The types of glenoid implants that may be included in a trial vector may be limited to the types of glenoid implant that are in the set of filtered glenoid implant types (i.e., the remaining glenoid implant types) determined in step 800. In other words, parameter prediction unit 206 may generate the trial vectors such that the trial vectors include only glenoid implant types in the set of remaining glenoid implant types. In some examples, the surgical parameter values may include a glenoid implant size parameter that is limited to the determined size of the glenoid implant. In other words, parameter prediction unit 206 may generate the trial vectors such that the trial vectors include only glenoid implants having the determined size.

Parameter prediction unit 206 may determine input values based on the surgical parameter values in a current trial vector (804). The input values may include values that parameter prediction unit 206 uses in a cost function to determine a cost value for the trial vector. Parameter prediction unit 206 may use a set of one or more functions to calculate the input values based on the surgical parameter values in the trial vector and the anatomic parameters of the patient. In some examples, the functions are provided to parameter prediction unit 206 by the surgeon. In some examples, the functions are preconfigured. For example, the surgical parameter values may include a glenoid implant version angle and a rule may specify a function that translates a glenoid implant version angle to an input value such that glenoid implant version angles closer to 0° have greater values than angles further from 0°. For instance, in this example, the function may be equal to a maximum glenoid implant version angle minus an absolute value of the glenoid implant version angle.

In another example, parameter prediction unit 206 may determine a volume reamed when the surgical parameters of the trial vector are applied given the anatomic parameters of the patient, determine an area of no contact between the implant and the bone when the surgical parameters of the trial vector are applied given the anatomic parameters of the patient, determine an area of contact between the implant and a strong portion of the bone when the surgical parameters of the trial vector are applied given the anatomic parameters of the patient, determine an area of contact between the implant and a weak portion of the bone when the surgical parameters of the trial vector are applied given the anatomic parameters of the patient, determine a radius of the glenoid implant indicated by the surgical parameter of the trial vector given the anatomic parameters of the patient, and/or determine other input values based on the surgical parameters.

Furthermore, in the example of FIG. 8, parameter prediction unit 206 may determine a first preliminary cost value for the current trial vector based on the input values (806). Parameter prediction unit 206 may determine the first preliminary cost value for the trial vector based on a linear combination of the input values as shown in Equation (1), below:

$${C}_{21} = {\sum }_{i = 1}^{m} ( {a}_{i} {b}_{i , k} ) \qquad ( 1 )$$

In Equation (1), above, C₁ indicates the first preliminary cost value for the trial vector, i is an index of the input values, m is the number of input values, aᵢ is a scaling factor for input value i, bᵢ is input value i in combination of inputs k, and offset is an offset value.

In one example where parameter prediction unit 206 determines the first preliminary cost value for the trial vector based on a linear combination of the input values, parameter prediction unit 206 may determine the first preliminary cost value for the trial vector as:

C₁=a₁·V_{Reamed}+a₂·A_{NoSeating}−a₃·A_{StrongSeating}−a₄·A_{WeakSeating}+a₅·Rᵢₘₚₗₐₙₜ³·|α_{Version}|+a₆·Rᵢₘₚₗₐₙₜ³·|α_{inclination}|+P·Rᵢₘₚₗₐₙₜ²+a₇V_{AnchoragePerforation} (2)

In Equation (2), above, C₁ denotes the first preliminary cost value for the trial vector, a₁ through a₇ denote weight values. V_{Reamed} indicates a volume reamed when the surgical parameters of the trial vector are applied. A_{NoSeating} indicates an area of no contact between the implant and the bone when the surgical parameters of the trial vector are applied. A_{StrongSeating} indicates an area of contact between the implant and a strong portion of the bone when the surgical parameters of the trial vector are applied. A_{WeakSeating} indicates an area of contact between the implant and a weak portion of the bone when the surgical parameters of the trial vector are applied. Rᵢₘₚₗₐₙₜ indicates a radius of the glenoid implant indicated by the surgical parameter of the trial vector. V_{AnchoragePerforation} indicates whether an anchorage (e.g., peg) of a glenoid implant perforates a boundary of the scapula opposite the glenoid or would come too close to the boundary of the scapula. In some examples, to determine whether the anchorage would come too close to the boundary of the scapula, parameter prediction unit 206 may compare a position of the anchorage to a scaled-down model of the scapula and determine whether any portion of the anchorage passes through a boundary of the scaled-down model of the scapula opposite the glenoid. α_{Version} and α_{Inclination} are fixed values. α_{Version} and α_{Inclination} may be determined based on an analysis of cases.

In Equation (2), P indicates a penalty value applied if any of the surgical parameters of the trial vector (or input values) are not consistent with the surgeon preferred values. For example, the surgical parameters of the trial vector include a version parameter that may range from −15° (retroversion) to 15° (anteversion). In this example, the surgeon preference parameters may specify a maximum retroversion of 10° (i.e., a version of −10°). Hence, in this example, if the surgical parameters of the trial vector include a version parameter of −15°, parameter prediction unit 206 may set P equal to the penalty value (e.g., 100). Otherwise, if the version parameter is not outside of the range specified by the surgeon preference parameters, parameter prediction unit 206 may set P equal to a non-penalty value (e.g., 0).

In Equation (2), V_{Reamed}, A_{NoSeating}, A_{StrongSeating}, and A_{WeakSeating} are based on anatomic parameters of the patient. A portion of a bone may be considered weak if a density of the bone is below a specified threshold. In some examples, a portion of a bone may be considered strong if a density of the bone is above the specified threshold.

In addition to determining the first preliminary cost value for a set of input values, parameter prediction unit 206 may determine a second preliminary cost value for the current trial vector (808). The second preliminary cost value may serve as part of a coherence verification that may ensure that values of the surgical parameters in the trial vector are within reasonable ranges. In some examples, parameter prediction unit 206 may determine the second preliminary cost value for the trial vector based on differences between the surgical parameters of the trial vector and typical values of the surgical parameters. For instance, parameter prediction unit 206 may determine the second preliminary cost value for the set of input values as follows:

$${C}_{2} = {\sum }_{k = 0}^{n} {a}_{k} \cdot {( \frac{{x}_{k} - {mean}_{k}}{{stdDev}_{k}} )}^{2} \qquad ( 3 )$$

In Equation (3) above, C₂ denotes the second preliminary cost value, k is an index of a surgical parameter, n denotes the number of surgical parameters in the trial vector, aₖ denotes a weight for surgical parameter k, xₖ denotes a value of surgical parameter k in the trial vector, meanₖ indicates a mean of values of surgical parameter k in a body of cases in which the surgery was previously performed, stdDevₖ is a standard deviation of surgical parameter k in the body of cases in which the surgery was previously performed. Equation (3), above, is calculated in a logarithmic scale. In Equation (3), above, each of the surgical parameters in the trial vector is assumed to follow a naïve Bayesian Gaussian distribution. Thus, equation (3) may be equivalent to calculating, for each surgical parameter Vₖ in the trial vector, given the values of other parameters x₁ . . . xₙ in the trial vector accordingly to the following chain rule:

$$P ( V ) = P ( {x}_{1} , … , {x}_{n} ) = P ) {x}_{1} , … {x}_{n} \vert {mean}_{1} , … , {mean}_{n} , {stdDev}_{1} , … , {stdDev}_{n} ) P ( {mean}_{1} , … , {mean}_{n} , {stdDev}_{1} , … , {stdDev}_{n} ) ∝ P ( {x}_{1} \vert {mean}_{1} , {stdDev}_{1} ) * P ( {mean}_{1} ) * P ( {stdDev}_{1} ) * … * P ( {X}_{n} \vert {mean}_{n} , {stdDev}_{n} ) * P ( {mean}_{n} ) * P ( {stdDev}_{n} ) ∝ P ( {x}_{1} \vert {mean}_{1} , {stdDev}_{1} ) * … * P ( {x}_{n} \vert {mean}_{n} , {stdDev}_{n} )$$

In other examples, it may be assumed that the surgical parameters in the trial vector follow other distributions. In some examples, different distributions may be assumed for different surgical parameters. For instance, seating percentage is an example of a surgical parameter. Many surgeons prefer the seating percentage to be 100%, but 100% seating is often not attainable in some patients. As a result, the mean seating percentage across patients (and hence a mean of the distribution for the seating percentage surgical parameter) may be less than 100% (e.g., 95%). Therefore, in this example, a different distribution from the distribution of derived directly from seating percentages in the trial vector may be used. For instance, a bias term may be used to modify the distribution derived from the seating percentages such that a mean of the distribution is equal to 100%.

Furthermore, in the example of FIG. 8, parameter prediction unit 206 may determine a cost value for the current trial vector based on the first preliminary cost value and the second preliminary cost value (810). For example, parameter prediction unit 206 may determine the cost value for the trial vector as:

C=(C₁+M)*C₂ (4)

In Equation (4), above, C denotes the cost value for the trial vector. C₁ is the first preliminary cost value, C₂ is the second preliminary cost value, and M is a constant that ensures that (C₁+M) and C₂ have the same sign.

Parameter prediction unit 206 may determine whether the cost value for the trial vector is less than a cost value for a previous trial vector (812). If the cost value for the trial vector is not less than the cost value for a previous trial vector (“NO” branch of 812), parameter prediction unit 206 may revert the trial vector to the previous trial vector (814). If the cost value for the current trial vector is less than the cost value for the previous trial vector (“YES” branch of 812) or after reverting the current trial vector to the previous trial vector, parameter prediction unit 206 may determine whether a stopping condition is met (816). In various examples, the stopping condition may be one or more of a specific number of times that step (816) has been reached, a particular number of times that a lower cost value has not been found, after evaluating all glenoid implant types in the remaining set of glenoid implant types, etc.

In response to determining that the stopping condition has not been met (“NO” branch of 816), parameter prediction unit 206 may generate a new trial vector (818) and repeat steps (804)-(816). Parameter prediction unit 206 may generate the new trial vector by updating one (or more) of the surgical parameter values of the current (or reverted) trial vector. For example, parameter prediction unit 206 may generate the new trial vector by incrementing or decrementing a surgical parameter value, such as the inclination angle, version angle, etc. In some examples, parameter prediction unit 206 may increment or decrement different surgical parameters by different amounts when generating new trial vectors. In another example, parameter prediction unit 206 may generate a new trial vector by changing a glenoid implant type to another glenoid implant type in the filtered set of glenoid implant types.

In this way, parameter prediction unit 206 may determine, based on a comparison of the cost value for a current trial vector in the set of trial vectors and a cost value of a previous trial vector in the set of trial vectors, whether the current trial vector represents an improvement over the previous trial vector. Additionally, based on the current trial vector not representing an improvement over the previous trial vector (e.g., the cost score for the current trial vector is less than the cost score for the previous trial vector), parameter prediction unit 206 may revert the current trial vector to the previous trial vector and update one or more surgical parameters of the current trial vector to determine a new current trial vector in the set of trial vectors. Alternatively, based on the current trial vector representing an improvement over the previous trial vector, parameter prediction unit 206 does not revert the current trial vector to the previous trial vector. In either case, parameter prediction unit 206 may generate a new trial vector based on the current trial vector.

After generating the new trial vector, parameter prediction unit 206 may repeat steps (804) through (816) with the new trial vector serving as the current trial vector. In this way, parameter prediction unit 206 may act as an amoeba optimizer that loops through combinations of implants and other surgical parameters. Thus, parameter prediction unit 206 may learn potentially optimal suggested surgical options for a specific patient based on the surgeon preference parameters.

In this way, parameter prediction unit 206 may generate a set of one or more trial vectors, where each trial vector in the set of trial vectors includes one or more of the surgical parameters. For each trial vector in the set of trial vectors, parameter prediction unit 206 may determine input values based on the surgical parameters of the trial vector and the anatomic parameters, and determine a cost value for the trial vector based on the input values.

On the other hand, if the stopping condition is met (“YES” branch of 816), parameter prediction unit 206 may determine a best trial vector in the set of trial vectors (820). The best trial vector is the trial vector having a lowest cost value when the stopping condition is met.

Each of the evaluated trial vectors in FIG. 8 may correspond to a different suggested surgical option. In some examples, parameter prediction unit 206 may rank the trial vectors based on their cost values and set a specific number of the trial vectors with the lowest cost values as the suggested surgical options. In some examples, parameter prediction unit 206 may determine, based on the cost value for a trial vector, whether to include the trial vector as one of the suggested surgical options. For instance, parameter prediction unit 206 may determine whether to include a trial vector as one of the suggested surgical options based on determining that the cost value for the trial vector exceeds a threshold.

Certain techniques of this disclosure are described with respect to a shoulder arthroplasty surgery and particularly with respect to a human scapula. Examples of shoulder arthroplasties include, but are not limited to, reversed arthroplasty, augmented reverse arthroplasty, standard total shoulder arthroplasty, augmented total shoulder arthroplasty, and hemiarthroplasty. However, the techniques are not so limited, and the visualization system may be used to provide virtual guidance information, including virtual guides in any type of surgery. Other example procedures in which surgical assistance system 100 may be used to provide virtual guidance include, but are not limited to, other types of orthopedic surgeries; any type of procedure with the suffix “plasty,” “stomy,” “ectomy,” “clasia,” or “centesis,”; orthopedic surgeries for other joints, such as elbow, wrist, finger, hip, knee, ankle or toe, or any other orthopedic surgery in which precision guidance is desirable. For instance, surgical assistance system 100 may be used to provide computer-assisted planning for an ankle arthroplasty surgery.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention. Moreover, techniques of this disclosure have generally been described with respect to human anatomy. However, the techniques of this disclosure may also be applied to animal anatomy in veterinary cases.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM. CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware.U Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

1: A method comprising:
obtaining, by a computing system, one or more surgeon preference parameters that specify values of one or more surgical parameters, wherein the surgical parameters include one or more positioning parameters for a glenoid implant to be attached to a glenoid fossa of a patient during a surgery;
determining, by the computing system, based on one or more anatomic parameters of the patient and the surgeon preference parameters, one or more suggested surgical options, each of the surgical options corresponding to a different combination of the positioning parameters for the glenoid implant and types of the glenoid implant; and
outputting, by the computing system, the one or more suggested surgical options.

2: The method of claim 1, wherein determining the one or more suggested surgical options comprises:
generating a set of one or more trial vectors, wherein each trial vector in the set of trial vectors includes one or more of the surgical parameters; and
for each trial vector in the set of trial vectors:
determining input values based on the surgical parameters of the trial vector and the anatomic parameters;
determining a cost value for the trial vector based on the input values; and
determining, based on the cost value for the trial vector, whether to include the trial vector as one of the suggested surgical options.

3: The method of claim 2, wherein determining the cost value for the trial vector comprises:
determining a first preliminary cost value for the trial vector based on a linear combination of the input values;
determining a second preliminary cost value for the trial vector based on differences between the surgical parameters of the trial vector and typical values of the surgical parameters; and
determining the cost value for the trial vector based on the first preliminary cost value for the trial vector and the second preliminary cost value for the trial vector.

4: The method of claim 2, wherein generating the set of trial vectors comprises:
filtering out glenoid implant types based on the surgeon preference parameters to determine a set of one or more remaining glenoid implant types; and
generating the trial vectors such that the trial vectors include only glenoid implant types in the set of remaining glenoid implant types.

5: The method of claim 2, wherein generating the set of trial vectors comprises:
determining a size of the glenoid implant based on the anatomical parameters of the patient; and
generating the trial vectors such that the trial vectors include only glenoid implants having the determined size.

6: The method of claim 2, wherein generating the set of trial vectors comprises generating a new trial vector in the set of trial vectors by updating one or more surgical parameters of the current trial vector.

7: The method of claim 2, wherein generating the set of trial vectors comprises:
determining, based on a comparison of the cost value for a current trial vector in the set of trial vectors and a cost value of a previous trial vector in the set of trial vectors, whether the current trial vector represents an improvement over the previous trial vector;
based on the current trial vector not representing an improvement over the previous trial vector, reverting the current trial vector to the previous trial vector; and
updating one or more surgical parameters of the current trial vector to determine a new current trial vector in the set of trial vectors.

8: The method of claim 2, wherein determining whether to include the trial vector as one of the suggested surgical options comprises determining whether the cost value for the trial vector exceeds a threshold.

9: The method of claim 1, wherein determining the one or more suggested surgical options comprises:
filtering glenoid implant types based on the surgeon preference parameters to determine a set of one or more remaining glenoid implant types;
determining, based on the anatomic parameters of the patient, a size of the glenoid implant;
generating a current trial vector that includes one or more surgical parameters, wherein a type of glenoid implant in the current trial vector is limited to the remaining glenoid implant types;
determining input values based on the surgical parameters of the current trial vector and the anatomic parameters;
determining a first preliminary cost value for the current trial vector based on a linear combination of the input values;
determining a second preliminary cost value for the current trial vector based on differences between the surgical parameters of the current trial vector and typical values of the surgical parameters; and
determining the cost value for the current trial vector based on the first preliminary cost value for the current trial vector and the second preliminary cost value for the current trial vector;
determining, based on a comparison of the cost value for the current trial vector and a cost value of a previous trial vector, whether the current trial vector represents an improvement over the previous trial vector;
based on the current trial vector not representing an improvement over the previous trial vector, reverting the current trial vector to the previous trial vector; and
updating one or more surgical parameters of the current trial vector to determine a new current trial vector.

10: A computing system comprising:
a memory configured to store one or more surgeon preference parameters that specify values of one or more surgical parameters, wherein the surgical parameters include one or more positioning parameters for a glenoid implant to be attached to a glenoid fossa of a patient during a surgery;
one or more processors implemented in circuitry, the one or more processors configured to:
determine, based on one or more anatomic parameters of the patient and the surgeon preference parameters, one or more suggested surgical options, each of the surgical options corresponding to a different combination of the positioning parameters for the glenoid implant and types of glenoid implant; and
output, for display, the one or more suggested surgical options.

11: The computing system of claim 10, wherein the one or more processors are configured such that, as part of determining the one or more suggested surgical options, the one or more processors:
generate a set of one or more trial vectors, wherein each trial vector in the set of trial vectors including one or more of the surgical parameters; and
for each trial vector in the set of trial vectors:
determine input values based on the surgical parameters of the trial vector and the anatomic parameters;
determine a cost value for the trial vector based on the input values; and
determine, based on the cost value for the trial vector, whether to include the trial vector as one of the suggested surgical options.

12: The computing system of claim 11, wherein the one or more processors are configured such that, as part of determining the cost value for the trial vector, the one or more processors:
determine a first preliminary cost value for the trial vector based on a linear combination of the input values;
determine a second preliminary cost value for the trial vector based on differences between the surgical parameters of the trial vector and typical values of the surgical parameters; and
determine the cost value for the trial vector based on the first preliminary cost value for the trial vector and the second preliminary cost value for the trial vector.

13: The computing system of claim 11, wherein the one or more processors are configured such that, as part of generating the set of trial vectors, the one or more processors:
filter out glenoid implant types based on the surgeon preference parameters to determine a set of one or more remaining glenoid implant types; and
generate the trial vectors such that the trial vectors include only glenoid implant types in the set of remaining glenoid implant types.

14: The computing system of claim 11, wherein the one or more processors are configured such that, as part of generating the set of trial vectors, the one or more processors:
determine a size of the glenoid implant based on the anatomical parameters of the patient; and
generate the trial vectors such that the trial vectors include only glenoid implants having the determined size.

15: The computing system of claim 11, wherein generating the set of trial vectors comprises generating a current trial vector by updating one or more surgical parameters of a previous trial vector in the set of trial vectors.

16: The computing system of claim 11, wherein the one or more processors are configured such that, as part of generating the set of trial vectors, the one or more processors:
determine, based on a comparison of the cost value for a current trial vector in the set of trial vectors and a cost value of a previous trial vector in the set of trial vectors, whether the current trial vector represents an improvement over the previous trial vector;
based on the current trial vector not representing an improvement over the previous trial vector, revert the current trial vector to the previous trial vector; and
update one or more surgical parameters of the current trial vector to determine a new current trial vector in the set of trial vectors.

17: The computing system of claim 11, wherein the one or more processors are configured such that, as part of determining whether to include the trial vector as one of the suggested surgical options, the one or more processors determine whether the cost value for the trial vector exceeds a threshold.

18: The computing system of claim 10, wherein the one or more processors are configured such that, as part of determining the one or more suggested surgical options, the one or more processors:
filter glenoid implant types based on the surgeon preference parameters to determine a set of one or more remaining glenoid implant types;
determine, based on the anatomic parameters of the patient, a size of the glenoid implant;
generate a current trial vector that includes one or more surgical parameters, wherein a type of glenoid implant in the current trial vector is limited to the remaining glenoid implant types;
determine input values based on the surgical parameters of the current trial vector and the anatomic parameters;
determine a first preliminary cost value for the current trial vector based on a linear combination of the input values;
determine a second preliminary cost value for the current trial vector based on differences between the surgical parameters of the current trial vector and typical values of the surgical parameters; and
determine the cost value for the current trial vector based on the first preliminary cost value for the current trial vector and the second preliminary cost value for the current trial vector;
determine, based on a comparison of the cost value for the current trial vector and a cost value of a previous trial vector, whether the current trial vector represents an improvement over the previous trial vector;
based on the current trial vector not representing an improvement over the previous trial vector, revert the current trial vector to the previous trial vector; and
update one or more surgical parameters of the current trial vector to determine a new current trial vector.

19. (canceled)

20: A non-transitory computer-readable storage medium having instructions stored thereon that when executed cause one or more processors of a computing system to:
obtain one or more surgeon preference parameters that specify values of one or more surgical parameters, wherein the surgical parameters include one or more positioning parameters for a glenoid implant to be attached to a glenoid fossa of a patient during a surgery;
determine, based on one or more anatomic parameters of the patient and the surgeon preference parameters, one or more suggested surgical options, each of the surgical options corresponding to a different combination of the positioning parameters for the glenoid implant and types of the glenoid implant; and
output the one or more suggested surgical options.

21: The non-transitory computer-readable storage medium of claim 20, wherein the instructions that cause the one or more processors to determine the one or more suggested surgical options include instructions that cause the one or more processors to:
generate a set of one or more trial vectors, wherein each trial vector in the set of trial vectors includes one or more of the surgical parameters; and
for each trial vector in the set of trial vectors:
determine input values based on the surgical parameters of the trial vector and the anatomic parameters;
determine a cost value for the trial vector based on the input values; and
determine, based on the cost value for the trial vector, whether to include the trial vector as one of the suggested surgical options.
